(19) United States
(12) Patent Application Publication
(10) Pub. No.: US 2021/0251234 A1
(43) Pub. Date: Aug. 19, 2021

(54) USE OF ISOTIANIL FOR CONTROL OF ZEBRA CHIP DISEASE

(71) Applicant: Bayer CropScience Aktiengesellschaft, Monheim am Rhein (DE)

(72) Inventors: Dinesh KUMAR, Duesseldorf (DE); Christoph Andreas BRAUN, Carmichael, CA (US); Juan Carlos TERRAZAS PORTILLO, Saltillo (MX); Elias TAPIA RAMOS, Cuautitlan Izcalli (MX); Luis Martin CAMBERO RAMIREZ, Cuautitlan Izcalli (MX)

(21) Appl. No.: 17/245,500

(22) Filed: Apr. 30, 2021

Related U.S. Application Data
(63) Continuation of application No. 15/763,864, filed on Mar. 28, 2018.

(30) Foreign Application Priority Data
Sep. 30, 2015 (EP) 15187800.6

(51) Int. Cl. A01N 43/80; A23L 19/18; A23L 19/12; C07D 275/03

(52) U.S. Cl. CPC A01N 43/80; C07D 275/03; A23L 19/12; A23L 19/18

(57) ABSTRACT

The present invention relates to the novel use of an isothiazolecarboxamide of the formula (I)

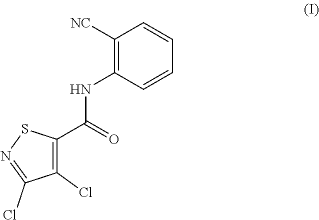 [SMILES: N#Cc1ccccc1NC(=O)c1snc(Cl)c1Cl]

(common name: Isotianil)
for controlling “Candidatus” Liberibacter spp and/or “Candidatus” Phytoplasma spp, preferably, “Candidatus” Liberibacter solanacearum and/or “Candidatus” Phytoplasma americanum, more preferred “Candidatus” Liberibacter solanacearum, in plants of the Solanaceae family, preferably in potatoes.

### CROSS-REFERENCE TO RELATED APPLICATIONS

This application is a continuation application of U.S. patent application Ser. No. 15/763,864, filed Mar. 28, 2018, which is a National Stage entry of International Application No. PCT/EP2016/073096, filed Sep. 28, 2016, which claims priority to European Patent Application No. 15187800.6, filed Sep. 30, 2015. Each of these applications is incorporated by reference in its entirety.

### BACKGROUND

### Field

The present invention relates to the novel use of an isothiazolecarboxamide of the formula (I)

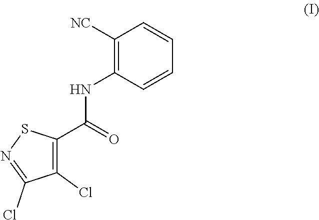 [SMILES: N#Cc1ccccc1NC(=O)c1snc(Cl)c1Cl]

(common name: Isotianil)
for controlling “Candidatus” Liberibacter spp and/or “Candidatus” Phytoplasma spp, preferably, “Candidatus” Liberibacter solanacearum and/or “Candidatus” Phytoplasma americanum, more preferred “Candidatus” Liberibacter solanacearum, in plants, preferably the plant is selected from the Solanaceae family, preferably the plant is a potato (Solanum tuberosum).

Moreover, the present application relates to a method of growing potatoes comprising a treatment regimen according to the present invention.

Furthermore, the present invention relates the use of Isotinail in accordance with the present invention as plant growth regulators for tuberous crop plants, preferably as plant growth regulators for potatoes, thereby increasing harvest yields tuberous crop plants (in particular the weight of the tuberous root) and/or increasing the plant growth of the tuberous root crop plants (in particular the growth of the leaves of the tuberous root crop plants), in each case in comparison to tuberous root crop plants not treated with agrochemically active compounds (untreated control), or alternatively, compared to standard treatment.

The present invention, moreover, relates to a reduction of the incidence of stained tubers in potatoes due to infection with “Candidatus” Liberibacter spp, in particular “Candidatus” Liberibacter solanacearum.

Further, the present invention relates to potato chips produced by frying potatoes obtained by the method according to the present invention.

Lastly, the present invention relates to a method of controlling said bacterial pathogens in plants of the Solanaceae family, in particular potatoes, by treating them with Isotianil.

### Description of Related Art

Candidatus spp are bacteria transmitted to potatoes and other solanaceous crops by psyllid vectors (e.g. Bactericera cockerelli).

Zebra chip disease is caused by Candidatus Liberibacter solanacearum which leads to brown or black stains on fried potato chips, thus decreasing the quality of the chips and, in worst case, preventing the use of the potatoes for this use at all.

Purple top disease is caused by Candidatus Phytoplasma americanum.

As there is no suitable way of antibiotical treatment of the plants, protection against the disease is typically obtained with an insecticide program aimed at controlling vectors.

However, the insecticide program still allows a high number of infections and, in particular, produces tubers which are upon frying show significant burning (blackening).

Thus, there is need to provide a method of treatment to improve the protection of potato plants against the bacterial infection and to prevent burning (blackening) of chips produced from infected potatoes.

### SUMMARY

It has now been found that Isotianil is particularly suitable for controlling “Candidatus” Liberibacter spp and/or “Candidatus” Phytoplasma spp, preferably, “Candidatus” Liberibacter solanacearum and/or “Candidatus” Phytoplasma americanum in plants of the Solanceae family, in particular potatoes.

A first subject matter of the invention is therefore the use of Isotianil for controlling said bacterial pathogenes in plants of the Solanceae family family.

A further subject matter of the invention is a method of controlling said bacterial pathogens in plants of the Solanaceae family, characterized in that the plants of the So family are treated with a compound selected from the compounds according to formula (I).

A further subject matter of the invention is a method of controlling said bacterial pathogens in plants of the Solanaceae family, characterized in that the plants of the Solanaceae family are treated with Isotianil simultaneously and/or in addition to the usual/standard treatment, wherein the as standard treatment application of insecticides is preferred.

### DETAILED DESCRIPTION OF A PREFERRED EMBODIMENT

Isotianil can therefore be employed for protecting plants against attack by the above mentioned pathogens and improving quality of tubers and chips produced therefrom by treatment of the plants/tubers upon planting and after emergence in regular intervals.

The intervals are from 2 to 20 days, preferably from 3 to 15 days, and more preferred from 4 to 14 days. In a preferred embodiment during the growth phase 5 to 20, preferably 6 to 11 treatments take place.

In a preferred embodiment the application rate is from 50 g ai/ha to 250 g ai/ha, preferably from 75 g ai/ha to 225 g ai/ha, more preferred from 100 g ai/ha to 200 g ai/ha, and most preferred 200 g ai/ha (g ai/ha=active ingredient per hectar, referring to Isotianil).

In a preferred embodiment the first treatment on planting is a soil application, while further treatments are foliar applications.

Alternatively, instead of a soil application, the seedling may be coated with the active compounds.

For an effizient control of the vector each treatment with isotianil is combined with a treatment of an insecticide, wherein the insecticide may be chosen according to severity of infestation, climate conditions, regulatory requirements and time of application. Useful insecticides for example are selected from the group comprising Imidacloprid, Spirotetramat, Spiromesifen and Spinetoram.

The good plant tolerance of Isotianil at the concentrations required for controlling plant diseases permits a treatment of aerial and subterranean plant parts, of vegetative propagation material, and of the soil.

Isotianil is also suitable for increasing the yield, show low toxicity and are well tolerated by plants.

Another embodiment of the present invention are potato chips produced from potatoes obtained according to the present invention which show less flake burning severity (blackening), thus having a better quality.

The present invention further comprises a method for producing potato chips comprising the steps of applying Isotianil upon seeding and after emergence in several treatments, preferably in combination with an insecticide, for controlling “Candidatus” Liberibacter solanacearum and/or “Candidatus” Phytoplasma americanum, harvesting the potatoes, cleaning, processing and cutting the potatoes, and frying the potatoes to obtain chips.

In the context of the present invention, an advantageous effect when applied to plants of the Solanaceae family was observed.

In accordance with the invention, all plants of the Solanaceae family may be treated. Plants of the Solanaceae family are, in the present context, understood as meaning all plant parts and plant populations, such as desired and undesired wild plants or crop plants (including naturally occurring crop plants). Crop plants may be plants of the Solanaceae family which can be obtained by traditional breeding and optimization methods or else by biotechnological and recombinant methods, or combinations of these methods, including the transgenic plants of the Solanaceae family and including the plant varieties capable or not of being protected by Plant Breeders' Rights. Plant parts are intended to mean all aerial and subterranean parts and organs of the plants, such as herb, pseudostem, shoot, leaf, bract, leaf sheaths, petiole, lamina, flower and root, examples which may be mentioned being leaves, needles, stalks, stems, flowers, fruiting bodies, fruit, bunches and seeds, and also roots, tubers, rhizomes, offshoots, suckers, secondary growth. The plant parts also include crop material and vegetative and generative propagation material, for example cuttings, tubers, rhizomes, slips and seeds.

As has already been mentioned above, all plants of the Solanaceae family can be treated in accordance with the invention. In a preferred embodiment, plant species and plant varieties, and their parts, which are found in the wild or which are obtained by conventional biological breeding methods, such as hybridization, meristem cultures, micropropagation, somatic embryogenesis, direct organogenesis or protoplast fusion, are treated. In a further preferred embodiment, transgenic plants of the Solanaceae family and plant varieties of the Solanaceae family which have been obtained by recombinant methods, if appropriate in combination with traditional methods (genetically modified organisms), are treated, such as, for example, transformation by means of Agrobacterium or particle bombardment of embryogenic cells, and micropropagation. Plants of the Solanaceae family include all plant parts as mentioned above.

It is especially preferred to treat, in accordance with the invention, plants of the Solanaceae family of those plant varieties which are in each case commercially available or in use. Plant varieties are understood as meaning plants with new properties (“traits”) which have been obtained by conventional breeding, by mutagenesis or else by recombinant DNA techniques. They may be varieties, breeds, biotypes and genotypes.

Depending on the plant species or plant varieties, their location and growth conditions (soils, climate, vegetation period, nutrition), the treatment according to the invention may also result in superadditive (“synergistic”) effects. Thus, for example, reduced application rates and/or extensions of the activity spectrum and/or an increase in the activity of the substances and compositions that can be used according to the invention, better plant growth, increased tolerance to high or low temperatures, increased tolerance to drought or to water or soil salinity, increased flowering performance, easier harvesting, accelerated maturation, higher yields, better quality and/or a higher nutritional value of the harvested crops, better storability and/or processability of the harvested crops, which exceed the effects which are actually to be expected, are possible.

The treatment method according to the invention can be used for the treatment of genetically modified organisms (GMOs), for example plants or seeds. Genetically modified plants (or transgenic plants) are plants in which a heterologous gene has been integrated stably into the genome. Essentially, the term “heterologous gene” refers to a gene which is provided or assembled outside the plant and which, upon introduction into the nuclear genome, the chloroplast genome or the mitochondrial genome of the transformed plant, confers novel or improved agronomical or other properties by expressing a protein or polypeptide of interest, or by downregulating or switching off another gene, or other genes, present in the plant (for example by means of antisense technology, cosuppression technology or RNAi technology [RNA interference]). A heterologous gene which is present in the genome is also referred to as a transgene. A transgene which is defined by its specific presence in the plant genome is referred to as transformation event, or transgenic event.

Depending on the plant species or plant varieties, their location and their growth conditions (soils, climate, vegetation period, nutrition), the treatment according to the invention may also result in superadditive (“synergistic”) effects. For example, the following effects are possible, which extend beyond the effects which are actually to be expected: reduced application rates and/or a widened spectrum of action and/or an increased efficacy of the active substances and compositions which can be employed in accordance with the invention, better plant growth, increased tolerance to high or low temperatures, increased tolerance to drought or water or soil salinity, improved flowering performance, easier harvesting, accelerated maturation, higher yields, larger fruit, greater plant height, more intensive green colour of the leaf, earlier flowering, better quality and/or higher nutritional value of the harvested crops, higher sugar concentration in the fruits, better storability and/or processability of the harvested crops.

Plants and plant varieties of the Solanaceae family which are preferably treated in accordance with the invention include all plants which contain hereditary material which confers especially advantageous, useful traits to these plants (no matter whether this has been achieved by breeding and/or biotechnology).

Plants and plant varieties of the Solanaceae family which are also preferably treated in accordance with the invention are resistant to one or more biotic stress factors, i.e. these plants have an improved defence against animal and microbial pathogens such as nematodes, insects, mites, phytopathogenic fungi, bacteria, viruses and/or viroids. Those which must be mentioned by preference in this context are Solanaceae which are resistant to phytopathogenic fungi or viruses.

Plants and plant varieties of the Solanaceae family which can also be treated in accordance with the invention are those plants which are resistant to one or more abiotic stress factors. The abiotic stress conditions may include for example drought, low-temperature and high-temperature conditions, osmotic stress, water-logging, increased soil salinity, increased exposure to minerals, ozone conditions, intensive light conditions, limited availability of nitrogen nutrients, limited availability of phosphorus nutrients, or shade avoidance.

Plants and plant varieties of the Solanaceae family which can also be treated in accordance with the invention are those plants in which vaccines or therapeutic proteins are expressed heterologously. These include, for example, hepatitis B antigen.

Plants and plant varieties of the Solanaceae family which can also be treated in accordance with the invention are those plants which are characterized by improved yield characteristics. In these plants, an increased yield may be caused by, for example, improved plant physiology, improved plant growth and improved plant development, such as water utilization efficacy, water holding efficacy, improved nitrogen utilization, increased carbon assimilation, improved photosynthesis, improved seed vigour, and accelerated maturation. The yield may furthermore be influenced by improved plant architecture (under stress and nonstress conditions), among which early flowering, control of flowering for the production of hybrid seed, seedling vigour, plant size, internode number and distance, root growth, seed size, fruit size, pod size, pod number or ear number, number of seeds per pod or ear, seed biomass, increased seed filling, reduced seed shedding, reduced pod shatter, and standing power. Further yield-related traits include seed composition such as carbohydrate content, protein content, oil content and oil composition, nutritional value, reduction of antinutritional compounds, improved processability and improved storability.

Plants or plant varieties of the Solanaceae family (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a Petunia EPSPS, a Tomato EPSPS, or an Eleusine EPSPS. It can also be a mutated EPSPS. Glyphosate-tolerant plants can also be obtained by expressing a gene that encodes a glyphosate oxido-reductase enzyme. Glyphosate-tolerant plants can also be obtained by expressing a gene that encodes a glyphosate acetyltransferase enzyme. Glyphosate-tolerant plants can also be obtained by selecting plants containing naturally occurring mutations of the abovementioned genes.

Other herbicide-resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant of the glutamine synthase enzyme that is resistant to inhibition. One such efficient detoxifying enzyme is, for example, an enzyme encoding a phosphinothricin acetyltransferase (such as, for example, the bar or pat protein from Streptomyces species). Plants expressing an exogenous phosphinothricin acetyltransferase have been described.

Further herbicide-tolerant plants are also plants that are made tolerant to the herbicides inhibiting the enzyme hydroxyphenylpyruvate dioxygenase (HPPD). Hydroxyphenylpyruvate dioxygenases are enzymes that catalyse the reaction in which para-hydroxyphenylpyruvate (HPP) is transformed into homogentisate. Plants tolerant to HPPD inhibitors can be transformed with a gene encoding a naturally occurring resistant HPPD enzyme, or a gene encoding a mutated HPPD enzyme. Tolerance to HPPD inhibitors can also be obtained by transforming plants with genes encoding certain enzymes enabling the formation of homogentisate despite the inhibition of the native HPPD enzyme by the HPPD inhibitor. The tolerance of plants to HPPD inhibitors can also be improved by transforming plants with a gene encoding an enzyme prephenate dehydrogenase in addition to a gene encoding an HPPD-tolerant enzyme.

Further herbicide-resistant plants are plants that are made tolerant to acetolactate synthase (ALS) inhibitors. Known ALS inhibitors include, for example, sulphonylurea, imidazolinone, triazolopyrimidines, pyrimidinyloxy(thio)benzoates and/or sulphonylaminocarbonyltriazolinone herbicides. Different mutations in the ALS enzyme (also known as acetohydroxyacid synthase (AHAS)) are known to confer tolerance to different herbicides and groups of herbicides. The production of sulphonylurea-tolerant plants and imidazolinone-tolerant plants is described in international publication WO 1996/033270. Further sulphonylurea- and imidazolinone-tolerant plants are also described in for example WO 2007/024782.

Other plants which are tolerant to imidazolinone and/or sulphonylurea can be obtained by induced mutagenesis, selection in cell cultures in the presence of the herbicide, or by mutation breeding.

Plants or plant varieties of the Solanaceae family (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

Plants or plant varieties of the Solanaceae family (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress tolerance plants include:
a. Plants which contain a transgene capable of reducing the expression and/or the activity of the poly(ADP-ribose)polymerase (PARP) gene in the plant cells or plants.
b. Plants which contain a stress tolerance-enhancing transgene capable of reducing the expression and/or the activity of the PARG-encoding genes of the plants or plant cells.
c. Plants which contain a stress tolerance-enhancing transgene coding for a plant-functional enzyme of the nicotinamide adenine dinucleotide salvage biosynthesis pathway including nicotinamidase, nicotinate phosphoribosyltransferase, nicotinic acid mononucleotide adenyltransferase, nicotinamide adenine dinucleotide synthetase or nicotin amide phosphoribosyltransferase.

### Application Forms

The treatment according to the invention of the plants of the Solanaceae family and plant parts and of the propagation material with a compound selected from among the compounds according to formula (I) is carried out directly or by acting on their environment, habitat or store by the customary treatment methods, for example by dipping, spraying, atomizing, nebulizing, scattering, painting on, injecting.

In an especially preferred embodiment of the present invention, a compound according to formula (I) or its formulations is used for application in foliar application.

Depending on its respective physical and/or chemical properties, the compound selected from among the compound according to formula (I) can be converted into the customary formulations, such as solutions, emulsions, suspensions, powders, foams, pastes, granules, sachets, aerosols, microencapsulations in polymeric substances, and ULV cold- and hot-fogging formulations.

These formulations are prepared in a known manner, for example by mixing the compound according to formula (I) with extenders, that is to say liquid solvents, pressurized liquefied gases and/or solid carriers, optionally with the use of surfactants, that is emulsifiers and/or dispersants and/or foam formers. If water is used as the extender, it is possible for example also to use organic solvents as cosolvents. Liquid solvents which are suitable in the main are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons, such as cyclohexane or paraffins, for example mineral oil fractions, alcohols such as butanol or glycol, and their ethers and esters, ketones such as acetone, methyl ethyl ketone, methyl isobutyl ketone or cyclohexanone, strongly polar solvents such as dimethylformamide and dimethyl sulphoxide, and water, and also mineral, animal and vegetable oils such as, for example, palm oil or other plant seed oils. Liquefied gaseous extenders or carriers are understood as meaning those liquids which are gaseous at normal temperature and under normal pressure, for example aerosol propellants such as halohydrocarbons and butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules are: for example crushed and fractionated natural rocks such as calcite, pumice, marble, sepiolite, dolomite, and synthetic granules of inorganic and organic meals, and granules of organic material such as sawdust, coconut shells, maize cobs and tobacco stalks. Emulsifiers and/or foam formers which are suitable are: for example nonionic, cationic and anionic emulsifiers, such as polyoxyethylene fatty acid esters, polyoxyethylene fatty alcohol ethers, for example alkylaryl poly glycol ethers, alkylsulphonates, alkyl sulphates, arylsulphonates, and protein hydrolysates. Suitable dispersants are: for example, lignosulphite waste liquors and methylcellulose.

Adhesives such as carboxymethylcellulose, natural and synthetic polymers in the form of powders, granules or latices, such as gum arabic, polyvinyl alcohol, polyvinyl acetate, and natural phospholipids such as cephalins and lecithins, and synthetic phospholipids, may be used in the formulations. Further additives may be mineral and vegetable oils.

It is possible to use colorants such as inorganic pigments, for example iron oxide, titanium oxide, Prussian Blue, and organic dyestuffs, such as alizarin, azo and metal phthalocyanine dyestuffs, and trace nutrients, such as salts of iron, manganese, boron, copper, cobalt, molybdenum and zinc.

In general, the formulations contain between 0.1 and 95% by weight of active substance, preferably between 0.5 and 90%.

Isotianil can be used in accordance with the invention and can be converted into the customary formulations, such as solutions, emulsions, suspensions, powders, foams and ULV formulations.

These formulations are prepared in the known manner by mixing Isotianil with customary additives, such as, for example, customary extenders and also solvents or diluents, colorants, wetters, dispersants, emulsifiers, antifoams, preservatives, secondary thickeners, adhesives, gibberellins, mineral and vegetable oils, and also water.

Colorants which may be present in the formulations which can be used in accordance with the invention are all colorants which are customary for such purposes. In this context, both pigments, which are sparingly soluble in water, and dyes, which are soluble in water, may be used. Examples which may be mentioned are the colorants known by the names Rhodamin B, C.I. Pigment Red 112 and C.I. Solvent Red 1.

Wetters which may be present in the formulations which can be used in accordance with the invention are all substances which are customary for formulating agrochemical active substances and which promote wetting. Alkylnaphthalenesulphonates, such as diisopropyl- or diisobutylnaphtha-lenesulphonates, may preferably be used.

Suitable dispersants and/or emulsifiers which may be present in the formulations which can be used in accordance with the invention are all nonionic, anionic and cationic dispersants which are conventionally used for the formulation of agrochemical active substances. The following may be used by preference: nonionic or anionic dispersants or mixtures of nonionic or anionic dispersants. Suitable nonionic dispersants which may be mentioned are, in particular, ethylene oxide/propylene oxide block polymers, alkylphenol poly glycol ethers and tristyrylphenol poly glycol ethers and their phosphated or sulphated derivatives. Suitable anionic dispersants are, in particular, lignosulphonates, salts of polyacrylic acid, and arylsulphonate/formaldehyde condensates.

Antifoams which may be present in the formulations which can be used in accordance with the invention are all foam-inhibitor substances which are conventionally used for the formulation of agrochemical active substances. Silicone antifoams and magnesium stearate may be used by preference.

Preservatives which may be present in the formulations which can be used in accordance with the invention are all substances which can be employed for such purposes in agrochemical compositions. Examples which may be mentioned are dichlorophene and benzyl alcohol hemiformal.

Secondary thickeners which may be present in the formulations which can be used in accordance with the invention are all substances which can be employed for such purposes in agrochemical compositions. Cellulose derivatives, acrylic acid derivatives, xanthan, modified clays and highly disperse silica are preferably suitable.

Adhesives which may be present in the formulations which can be used in accordance with the invention are all customary binders which can be used in mordants. Polyvinylpyrrolidone, polyvinyl acetate, polyvinyl alcohol and tylose may be mentioned by preference.

Gibberellins which may be present in the formulations which can be used in accordance with the invention are preferably Gibberellin A1, Gibberellin A3 (gibberellic acid), Gibberellin A4, Gibberellin A7. Especially preferred is gibberellic acid.

The gibberellins are known (cf. R. Wegler “Chemie der Pflanzenschutz- and Schädlingsbekämpfungsmittel” [Chemistry of plant protection and pesticide agents], volume 2, Springer Verlag, Berlin-Heidelberg-New York, 1970, pages 401-412).

### Mixtures

Isotianil can be employed as such or, in formulations, also in a mixture with known fungicides, bactericides, acaricides, nematicides, herbicides, insecticides, safeners, soil-improvement products or products for reducing plant stress, for example Myconate, in order to widen the spectrum of action or to prevent the development of resistance, for example. In many cases, this engenders synergistic effects, that is to say the efficacy of the mixture exceeds the efficacy of the individual components.

In the present invention Isotianil is preferably used as a mixture with an insecticide.

In accordance with the invention, the term “mixture” means various combinations of at least two of the abovementioned active substances which are possible, such as, for example, ready mixes, tank mixes (which is understood as meaning spray slurries prepared from the formulations of the individual active substances by combining and diluting prior to the application) or combinations of these (for example, a binary ready mix of two of the abovementioned active substances is made into a tank mix by using a formulation of the third individual substance). According to the invention, the individual active substances may also be employed sequentially, i.e. one after the other, at a reasonable interval of a few hours or days, in the case of the treatment of seed for example also by applying a plurality of layers which contain different active substances. Preferably, it is immaterial in which order the individual active substances can be employed.

The compound according to formula (I) can be employed as such, in the form of its formulations or the use forms prepared therefrom, such as ready-to-use solutions, suspensions, wettable powders, pastes, soluble powders, dusts and granules. They are applied in the customary manner, for example by pouring, spraying, atomizing, scattering, dusting, foaming, painting on and the like. It is furthermore possible to apply the compound according to formula (I) by the ultra-low-volume method or to inject the active substance preparation, or the active substance itself, into the soil. The vegetative propagation material of the plants may also be treated.

The examples which follow are intended to illustrate the invention, but without imposing any limitation.

### EXAMPLES

The seeding was made on June 10. The potato variety used was Caesar, third-category tubercles were used. A soil application was made to all the treatments when fluoxastrobin fungicides were applied; 2-litters dose or commercial product per hectare, and penfuflen 0.650 litters dose per hectare; in order to prevent Fusarium spp. and/or Rhizoctoniasolani fungus attack. The experimental design used was random blocks with four repetitions; the experimental parcel was formed by four furrows at a distance between furrows of 0.92 m, and 10 meters length. The useful parcel consisted in two central furrows of each experimental parcel. Distance between plants was 0.25 m.

The applied treatments consisted in an application on the bottom of the furrow when the seeding was taking place, and eleven foliar applications. The treatments that have been applied are shown in Table 1.

TABLE 1
Applied treatments to determine isotianil efficiency and selectivity in bacterial disease.

| | | Treatments | | | | | |
|---|---|---|---|---|---|---|---|
| Application | Date | 1 | 2 | 3 | 4 | 5 | 6 |
| 1.- Seeding | 10/06/ | Untreated | Admire | ISOTIANIL | ISOTIANIL | Admire | Admire |
| | | | | 100 gia/ha* | 200 gia/ha* | ISOTIANIL | ISOTIANIL |
| | | | | | | 100 gia/ha* | 200 gia/ha* |
| 2.- Foliar | 02/07/ | Untreated | Movento | ISOTIANIL | ISOTIANIL | Movento | Movento |
| | | | | 100 gia/ha* | 200 gia/ha* | ISOTIANIL | ISOTIANIL |
| | | | | | | 100 gia/ha* | 200 gia/ha* |
| 3.- Foliar | 10/07/ | Untreated | Muralla | ISOTIANIL | ISOTIANIL | Muralla | Muralla |
| | | | max | 100 gia/ha* | 200 gia/ha* | ISOTIANIL | ISOTIANIL |
| | | | | | | 100 gia/ha* | 200 gia/ha* |
| 4.- Foliar | 14/07/ | Untreated | Movento | ISOTIANIL | ISOTIANIL | Movento | Movento |
| | | | | 100 gia/ha* | 200 gia/ha* | ISOTIANIL | ISOTIANIL |
| | | | | | | 100 gia/ha* | 200 gia/ha* |
| 5.-Foliar | 21/07/ | Untreated | Muralla | ISOTIANIL | ISOTIANIL | Muralla | Muralla |
| | | | max | 100 gia/ha* | 200 gia/ha* | ISOTIANIL | ISOTIANIL |
| | | | | | | 100 gia/ha* | 200 gia/ha* |
| 6.- Foliar | 31/07/ | Untreated | New | ISOTIANIL | ISOTIANIL | New | New |
| | | | leverage | 100 gia/ha* | 200 gia/ha* | Leverage | Leverage |
| | | | | | | ISOTIANIL | ISOTIANIL |
| | | | | | | 100 gia/ha* | 200 gia/ha* |
| 7.-Foliar | 05/08/ | Untreated | Oberon | ISOTIANIL | ISOTIANIL | Oberon | Oberon |
| | | | | 100 gia/ha* | 200 gia/ha* | ISOTIANIL | ISOTIANIL |
| | | | | | | 100 gia/ha* | 200 gia/ha* |
| 8.- Foliar | 12/08/ | Untreated | Oberon | ISOTIANIL | ISOTIANIL | Oberon | Oberon |
| | | | | 100 gia/ha* | 200 gia/ha* | ISOTIANIL | ISOTIANIL |
| | | | | | | 100 gia/ha* | 200 gia/ha* |
| 9.- Foliar | 18/08/ | Untreated | Exalt | ISOTIANIL | ISOTIANIL | Exalt | Exalt |
| | | | | 100 gia/ha* | 200 gia/ha* | ISOTIANIL | ISOTIANIL |
| | | | | | | 100 gia/ha* | 200 gia/ha* |
| 10.- Foliar | 02/09/ | Untreated | Movento | ISOTIANIL | ISOTIANIL | Movento | Movento |
| | | | | 100 gia/ha* | 200 gia/ha* | ISOTIANIL | ISOTIANIL |
| | | | | | | 100 gia/ha* | 200 gia/ha* |
| 11.- Foliar | 08/09/ | Untreated | Muralla | ISOTIANIL | ISOTIANIL | Muralla | Muralla |
| | | | max | 100 gia/ha* | 200 gia/ha* | ISOTIANIL | ISOTIANIL |
| | | | | | | 100 gia/ha* | 200 gia/ha* |
| 12.- Foliar | 19/09/ | Untreated | New | ISOTIANIL | ISOTIANIL | New | New |
| | | | leverage | 100 gia/ha* | 200 gia/ha* | Leverage | Leverage |
| | | | | | | ISOTIANIL | ISOTIANIL |
| | | | | | | 100 gia/ha* | 200 gia/ha* |

*gia/ha = grams of active ingredient per hectare

### Evaluations

Emergence: Cultivation emergence was determined; for which the number of plants that emerged in each parcel central furrows was counted at 100% emergence. The data were collected on Jul. 2, 2014 (22 days after seeding).

Phytotoxicity percentage was determined.

Bactericera cockerelli, Population Dynamic: Studies on adult psilides, nymphs and eggs were population dynamic were made. For which purpose a count was made before the first foliar application and subsequently after each treatment application. To know the number of adults 20 net traps (with entomologic net) per experimental parcel, in each evaluation. To determine the number of eggs and nymphs, 50 pinnas were evaluated per parcel in each evaluation.

Zebra chip/purple top Dynamic: Dynamic on zebra chip/purple top symptoms appearance was registered for each application date, for which incidence and severity was determined.

The tubercle staining damage was registered, for which 5 tubercles for each category were selected. (In some repetitions, the number of the first and second categories of tubercles was less than five, for which reason those obtained were evaluated) for each parcel. A cross cut was made to the center of each tubercle to determine the staining level. Incidence and severity were determined for which the scale described by Flores-Olivas 2013, was taken as basis. The burning damage or zebra chip in fried slices was also analyzed, similarly to the methodology used for tubercles.

Production: In harvest, each treatment production was analyzed, for which two lineal meters were harvested of each experimental parcel central furrows, registering the potato tubercles production for each of the four categories (first, second, third and combined).

TABLE 2
Treatment germination percentage.

| | Emergence |
|---|---|
| Treatment | Percentage (%) |
| 1 | 70.3 |
| 2 | 64.3 |
| 3 | 61.2 |
| 4 | 71.8 |
| 5 | 65.3 |
| 6 | 67.8 |

Population Dynamic on Bactericera cockerelli (Sulc).

Adults: From August 5 (55 days after seeding), B. cockerelli adults were captured basically in the witness treatment parcels, with an average of 7.5 adults per parcel (Table 3). In the evaluation dated August 12, a greatest amount of adults in all the parcels was observed, notwithstanding, in the witness treatment the amount of captured adults was greater than in the rest of the treatments. The population dynamic was decreasing on subsequent dates until achieving practically zero on September 08, and increase on the last evaluated dated which was September 19, with a drastic increase in adult population. As observed in Table 3 and Graphic 2, the number of captured adults was greater in the witness and lower in those treatments with insecticides (2, 5 and 6). These results indicate us the necessity of implement an insecticide application program based on an efficient monitoring of the insect in view that those treatments where insecticides were not applied including those where isotianil was applied, the adult insect presence was greater

TABLE 3
Four-repetitions average of registered adults
for each sampling date, for each treatment.

| | average of registered adults | | | | | |
|---|---|---|---|---|---|---|
| Treat. | 5 - Aug. | 12-Aug. | 18-Aug. | 02-Sep. | 08-sep. | 19-Sep. |
| T1 | 7.5 | 5.75 | 4.5 | 2.75 | 0 | 11.25 |
| T2 | 0 | 1.25 | 2 | 1 | 0 | 5 |
| T3 | 1 | 1.25 | 3.75 | 1.75 | 0.5 | 4.75 |
| T4 | 0.75 | 2 | 1 | 0.25 | 0 | 2.75 |
| T5 | 0 | 0.75 | 2 | 0 | 0 | 2.5 |
| T6 | 0 | 1 | 1.5 | 0 | 0 | 3 |

Nymphs: Regarding the presence of B. cockerelli immature stages, the greatest amount of nymphs was detected on September 02 (Table 4), which can coincide with the adult population top on August 18, and in turn generated the greatest adult population observed on September 19. Once again, it can be observed that in the treatments with insecticides, the number of nymphs was lower than in the witness treatment.

TABLE 4
Average of four repetitions of registered nymphs
for each sampling date, for each treatment.

| | Average of registered B.cokerelli nymphs | | | | | |
|---|---|---|---|---|---|---|
| Treat. | 5 - Aug. | 12-Aug. | 18-Aug. | 02-Sep. | 08-sep. | 19-Sep. |
| T1 | 0 | 5.25 | 2.5 | 20 | 8.5 | 0 |
| T2 | 0 | 0 | 0 | 1.5 | 8 | 0 |
| T3 | 0 | 0 | 0 | 4.5 | 3 | 0 |
| T4 | 0 | 2.25 | 0.25 | 13.75 | 0 | 0 |
| T5 | 0 | 0 | 0 | 2.25 | 6.75 | 0 |
| T6 | 0 | 0 | 0 | 0 | 0 | 0 |

TABLE 5
Average of four repetitions of registered eggs
for each sampling date, for each treatment.

| | B. Cokerelli eggs population average | | | | | |
|---|---|---|---|---|---|---|
| Treat. | 5 - August | 12-August | 18-August | 02-Sep. | 08-sep. | 19-Sep. |
| T1 | 0 | 9 | 0 | 4 | 1.25 | 0 |
| T2 | 0 | 3 | 0 | 0 | 3.25 | 0 |
| T3 | 0 | 1.5 | 0 | 0 | 1.25 | 0 |
| T4 | 0 | 4.5 | 0 | 0 | 0.75 | 0 |
| T5 | 0 | 0 | 0 | 0 | 0 | 0 |
| T6 | 0 | 0 | 0 | 2 | 0 | 0 |

As can be seen, the combined treatments of Insecticide and Isotianil provided the most effective protection.

TABLE 7
Zebra chip/purple top incidence control percentage
for each sampling date.

| | Control |
|---|---|
| Treat. | percentage |
| 1 | 0 |
| 2 | 66.3 |
| 3 | 14.35 |
| 4 | 62.65 |
| 5 | 73.63 |
| 6 | 92.91 |

Production.—Potato production data are shown in Table 8. Rreatment 6 was superior to the rest of the treatments, followed by treatments 2 and 5.

TABLE 8
Potato treatment production mean.
Total production in Kilograms per
parcel

| Treat. | Average |
|---|---|
| 1 | 18.475 |
| 2 | 36.2 |
| 3 | 17.975 |
| 4 | 21.3 |
| 5 | 31.675 |
| 6 | 43.3 |

The commercial quality analysis of harvested tubercles for each treatment shows again that treatment 6 generated the best yield in first and second quality categories, in comparison with the rest of the treatments.

TABLE 10
Production mean per treatment in each of the four
potato quality categories.
Production in Kilograms per treatment categories

| Treat. | First | Second | Third | Combined |
|---|---|---|---|---|
| 1 | 0 | 0 | 21.9 | 52.51 |
| 2 | 18.3 | 27.95 | 53.3 | 45.3 |
| 3 | 0 | 7.8 | 24.3 | 39.8 |
| 4 | 4 | 7.8 | 35.7 | 37.7 |
| 5 | 22.8 | 20.1 | 35.2 | 48.6 |
| 6 | 56.8 | 38.4 | 50.0 | 28 |

Zebra chip or tubercle staining.

TABLE 11
Statistical analysis of tubercle staining incidence and
severity, and percentage of the incidence and
severity control on zebra chip/purple top.

| | Incidence | Severity |
|---|---|---|
| | Control | Control |
| Treat. | Percentage | Percentage |
| 1 | 0 | 0 |
| 2 | 47.99 | 48.36 |
| 3 | 35.01 | 43.13 |
| 4 | 63.35 | 74.50 |
| 5 | 88.26 | 86.92 |
| 6 | 64.16 | 55.88 |

### Flake Burning Severity

| | Incidence |
|---|---|
| | control |
| Treat. | percentage |
| 1 | 0 |
| 2 | 31.7 |
| 3 | 31.3 |
| 4 | 54 |
| 5 | 74 |
| 6 | 72.1 |

As can be seen, treatments 5 and 6 provided the best results with regard to flake burning severity, i.e. blackening of the chips upon frying is significantly reduced.

1. A method of controlling “Candidatus” Liberibacter spp and/or “Candidatus” Phytoplasma spp in one or more plants of the Solanaceae family comprising treating the plants of the Solanaceae family with isotianil simultaneously with or in addition to an insecticide treatment.

2. The method according to claim 1, wherein the plant is potato (Solanum tuberosum).

3. The method according to claim 1, wherein the controlled pathogen is “Candidatus” Liberibacter solanacearum and/or “Candidatus” Phytoplasma americanum.

4. The method according to claim 1, wherein during the growth phase 5 to 20 treatments take place.

5. The method according to claim 1, wherein intervals between treatment are from 2 to 20 days.

6. The method according to claim 1, wherein intervals between treatment are 4 to 14 days.

7. The method according to claim 1, wherein an application rate of isotianil is from 50 g ai/ha to 250 g ai/ha.

8. The method according to claim 1, wherein an application rate of isotianil is from 75 g ai/ha to 225 g ai/ha.

9. The method according to claim 1, wherein an application rate of isotianil is from 100 g ai/ha to 200 g ai/ha.

10. The method according to claim 1, wherein the applied insecticide is selected from the group consisting of imidacloprid, spirotetramat, spiromesifen and spinetoram.

11. The method according to claim 1, wherein the applied insecticide is imidacloprid.

12. The method according to claim 1, wherein the applied insecticide is spirotetramat.

13. The method according to claim 1, wherein the applied insecticide is spiromesifen.

14. The method according to claim 1, wherein the applied insecticide is selected from the group consisting of spinetoram.

15. The method according to claim 1, wherein a first treatment is a soil application and all further treatments are foliar applications.

16. The method according to claim 2, further comprising producing potato chips from the treated potato chips, wherein said potato chips show less flake burning severity.

17. A method for producing potato chips comprising
applying isotianil upon seeding and after emergence in several treatments, optionally in combination with an insecticide, for controlling “Candidatus” Liberibacter solanacearum and/or “Candidatus” Phytoplasma americanum, harvesting the potatoes,
cleaning, processing and cutting the potatoes, and
frying the potatoes.
